(19) United States
(12) Patent Application Publication
(10) Pub. No.: US 2011/0152953 A1
(43) Pub. Date: Jun. 23, 2011

(54) SURGICAL GUIDE INSTRUMENT AND METHOD FOR WORKING THE ARTICULAR PROCESSES OF VERTEBRAL BODIES

(75) Inventor: Helmut D. Link, Hamburg (DE)

(73) Assignee: DERU GMBH, Norderstedt (DE)

(21) Appl. No.: 12/949,360

(22) Filed: Nov. 18, 2010

Related U.S. Application Data
(60) Provisional application No. 61/292,349, filed on Jan. 5, 2010.

(30) Foreign Application Priority Data
Nov. 20, 2009 (EP) 09014509.5

(51) Int. Cl. A61B 17/56

(52) U.S. Cl. 606/86 A

(57) ABSTRACT

A surgical guide instrument for use on the articular processes of vertebral bodies. At the distal end of the guide instrument, there are two plier heads, which are designed for a plier movement relative to each other. A guide rail is provided, which defines a guide axis for a reamer. The guide rail is pivotable about a first pivot axis, such that the guide axis, with the pivoting movement about the first pivot axis, spans a work sector, and such that the work sector intersects an imaginary connecting line between the plier heads.

### CROSS REFERENCE TO RELATED APPLICATIONS

The present application claims the benefit of European Application 09014509.5 filed 20 Nov. 2009 and U.S. Provisional Application Ser. No. 61/292,349 filed 5 Jan. 2010, each of which are incorporated herein by reference in their entireties.

### FIELD OF THE INVENTION

The disclosure relates to a surgical guide instrument for working the articular processes of vertebral bodies. The disclosure also relates to a method for working such articular processes.

### BACKGROUND

Prostheses for replacing the articular surfaces of facet joints are known in which a shaft of the prosthesis is received in a recess of the articular process (see EP 1 959 871). Such recesses in the bones are normally formed using a reamer. The reamer has hitherto been guided freely by the surgeon. However, important nerves and blood vessels are located near the operating site. An incautious movement on the part of the surgeon can lead to these being damaged.

The object is to make available a guide instrument for working the articular processes, and an associated method, with which the risk of accidental injury to the patient is avoided. The object is achieved by the features of the independent claims. Advantageous embodiments are set forth in the dependent claims.

### SUMMARY

According to one embodiment, two plier heads are arranged at a distal end of a surgical guide instrument, which plier heads are designed for a plier movement relative to each other. The guide instrument comprises a guide rail, which defines a guide axis for a reamer. The guide rail is pivotable about a first pivot axis, such that the guide axis, with the pivoting movement about the first pivot axis, spans a work sector, and such that the work sector intersects an imaginary connecting line between the plier heads.

The articular process that is to be worked is being engaged by the plier heads in order thereby to bring the guide instrument into a defined position relative to the articular process. The guide rail is thus likewise in a defined position relative to the articular process, and the freedom of movement of the guide rail is limited to pivoting movements relative to the guide instrument. In particular, in a pivoting movement about the first pivot axis, the guide axis of the guide rail sweeps across a work sector that corresponds to the work range of a reamer guided in the guide rail. This work sector intersects an imaginary connecting line between the plier heads, which is the same as saying that a reamer received in the guide rail can be guided through a space located between the plier heads. Since the plier heads hold the articular process between them, the movement of the reamer through the guide rail is limited to the area in which the articular process is to be worked. The risk of the patient being injured by an incautious movement on the part of the surgeon is reduced.

In an advantageous embodiment, the guide rail is pivotable about a second pivot axis. By pivoting about the second pivot axis, the orientation of the work sector between the plier heads can be modified. The area in which the reamer works the articular process can thus be brought closer to one plier head or to the other plier head. In this way, it is no longer necessary to position the plier heads absolutely precisely. Slight imprecision in the positioning of the plier heads can be compensated by the fact that the guide rail is pivoted about the second pivot axis and, in this way, the work sector is brought into the correct position between the plier heads.

The second pivot axis preferably lies in the plane of the work sector. This has the advantage that the orientation of the work sector does not change when the guide rail is pivoted about the second pivot axis.

The freedom of movement of the guide rail about the first pivot axis is normally limited by two stops. The stops also define the limits of the work sector. The extent of the work sector in degrees, which corresponds to the angle range, is preferably greater than 15°, more preferably greater than 30°, more preferably greater than 45°.

For the plier movement of the plier heads, a plier joint can be provided, which connects two plier limbs to each other. The plier limbs can extend from the plier heads to the plier joint and beyond the latter to a grip end of the guide instrument. The grip end is designated as the proximal end of the guide instrument, while the opposite end, on which the plier heads are arranged, is designated as the distal end. In an advantageous embodiment, the plier limbs between the plier joint and the plier heads are angled out from a plane oriented perpendicular to the plier joint. The plier heads thus lie outside the plane that is spanned by the grip ends of the plier limbs. This configuration makes it easier to engage the instrument round the rearwardly extending spinous processes of the vertebral bodies. The length of the angled part of the plier limbs preferably corresponds approximately to the spinous process. For operating the guide instrument, it is also expedient if, in relation to a plane lying perpendicular to the axis of the plier joint and extending through the plier joint, the plier heads are arranged on one side and the first pivot axis on the other side. In relation to a longitudinal axis extending through the plier heads, the plier joint is preferably arranged on one side and the first pivot axis on the other side.

A plurality of projections can be provided on the plier head, which projections are oriented in the direction of the plier movement. Therefore, when an articular process is engaged by the plier heads, the projections come into contact with the articular process first. The projections preferably taper to a point, such that they can easily penetrate into the bone tissue. When the projections penetrate into the bone tissue, they provide a better hold for the guide instrument.

When implanting a facet joint prosthesis, it is generally the case that both the superior articular surface and also the inferior articular surface are replaced. To do so, a recess for receiving the shaft of the prosthetic component has to be formed in both articular processes involved. The guide instrument opens up the possibility of forming the recess in both articular processes in a single combined work step. For this purpose, the articular processes can be engaged by the guide instrument in such a way that both articular processes are fixed between the plier heads. With a pivoting movement of the reamer about the first pivot axis, a recess can be formed that extends through both articular processes. For this purpose, the projections are preferably arranged on the plier head in such a way that at least one projection engages in each of the two articular processes. This can be achieved by means of the projections being arranged on both sides of a longitudinal axis extending through the plier head.

Another embodiment relates to an instrument set, which comprises a guide instrument as described above and a reamer. The reamer is characterized in that it has a shaft adapted to the guide rail. The shaft can be inserted into the guide rail, such that the direction of the reamer is defined when it rotates in the guide rail. The reamer can be movable in the longitudinal direction in relation to the guide rail. In this way, it is possible to form recesses of different depths in the articular processes. The length of the reamer is at least such that it extends from the guide rail to the imaginary connecting line between the plier heads.

An embodiment of the method provided herein relates to a method for working the articular processes of vertebral bodies. In said method, a pivotable guide rail is first positioned relative to the articular process, such that a work sector spanned by the pivoting movement of the guide rail intersects the articular process. The guide rail is then fixed relative to the articular process, such that the guide rail is limited to pivoting movements. The articular process is worked using a reamer inserted into the guide rail. The guide rail is for this purpose pivoted together with the reamer, such that the reamer works the articular process and forms a recess. The method can be carried out, for example, using the guide instrument as described above.

In one embodiment of the method, the guide rail can be fixed relative to two articular processes. The two articular processes, which preferably interact in one facet joint, can then be worked with the reamer in a common work step. The depth of penetration of the reamer into the articular process can be adjusted by moving the reamer along the guide rail. Further optional features of the method according to further embodiments derive from the above description of the operation of the guide instrument.

### BRIEF DESCRIPTION OF THE DRAWINGS

Exemplary embodiments are described below with reference to the attached drawings, in which:

FIG. 1 shows a detail of a lumbar region of the spinal column;

FIG. 2 shows a detail of cervical vertebral bodies with an implanted facet joint prosthesis;

FIG. 3 shows a detail of cervical vertebral bodies with another embodiment of a facet joint prosthesis;

FIG. 4 shows a guide instrument according to one embodiment;

FIG. 5 shows another view of the guide instrument from FIG. 4;

FIG. 6 shows the distal end of the guide instrument in a view corresponding to FIG. 5;

FIG. 7 shows an enlarged detail from FIG. 6; and

FIG. 8 shows the distal end of the guide instrument in a view corresponding to FIG. 4.

### DETAILED DESCRIPTION OF THE INVENTION

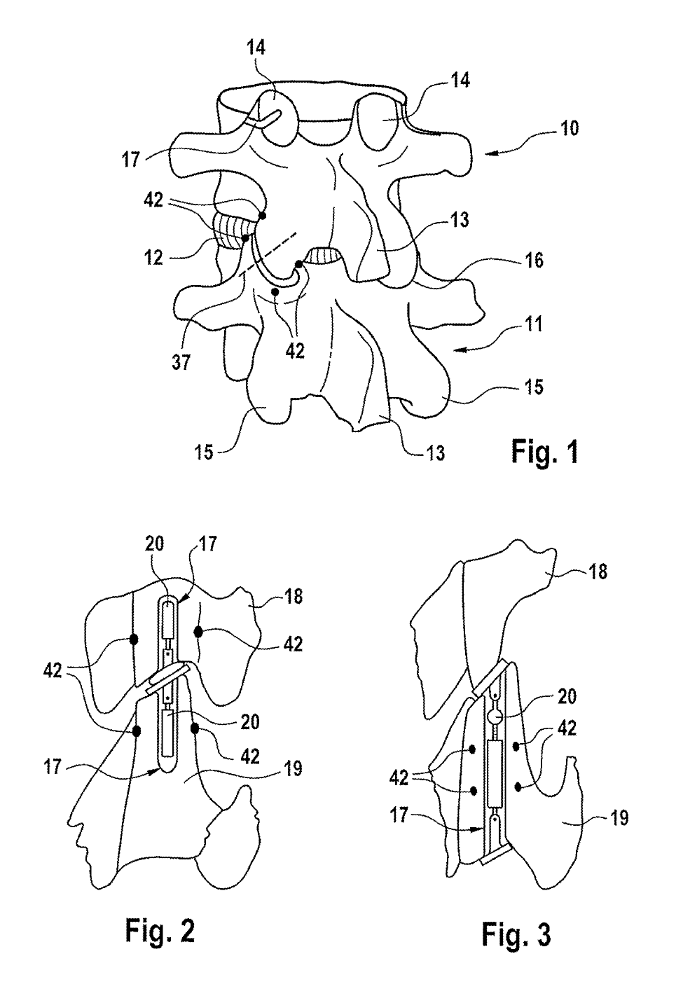

A detail of a human spinal column in FIG. 1 shows two lumbar vertebral bodies 10, 11 with an intervertebral disk 12 arranged between them, and each with a rearwardly extending spinous process 13. The vertebral bodies 10, 11 each comprise two superior articular processes 14 and two inferior articular processes 15. Between the two vertebral bodies 10, 11, the inferior articular processes of the upper vertebral body 10 and the superior articular processes of the lower vertebral body 11 interact and form two facet joints 16. During a movement of the vertebral bodies, the articular surfaces of the facet joints 16 perform a sliding movement relative to each other.

With a facet joint prosthesis (not shown in FIG. 1), the articular surfaces of the facet joints 16 are replaced by artificial articular surfaces. In order to secure the prosthesis, a shaft extends through the articular process, which is tensioned from the opposite side. In order to receive the shaft, recesses are formed in the articular processes. Such a recess 17 is indicated by way of example for the superior articular process 14 of the upper vertebral body 10.

Corresponding facet joint prostheses can also be used in the cervical region of the spinal column. FIGS. 2 and 3 each show details of the inferior articular processes 18 and superior articular processes 19 of two adjacent cervical vertebral bodies. Recesses 17 are formed in the articular processes 18, 19 and receive the shafts of different embodiments of facet joint prostheses 20.

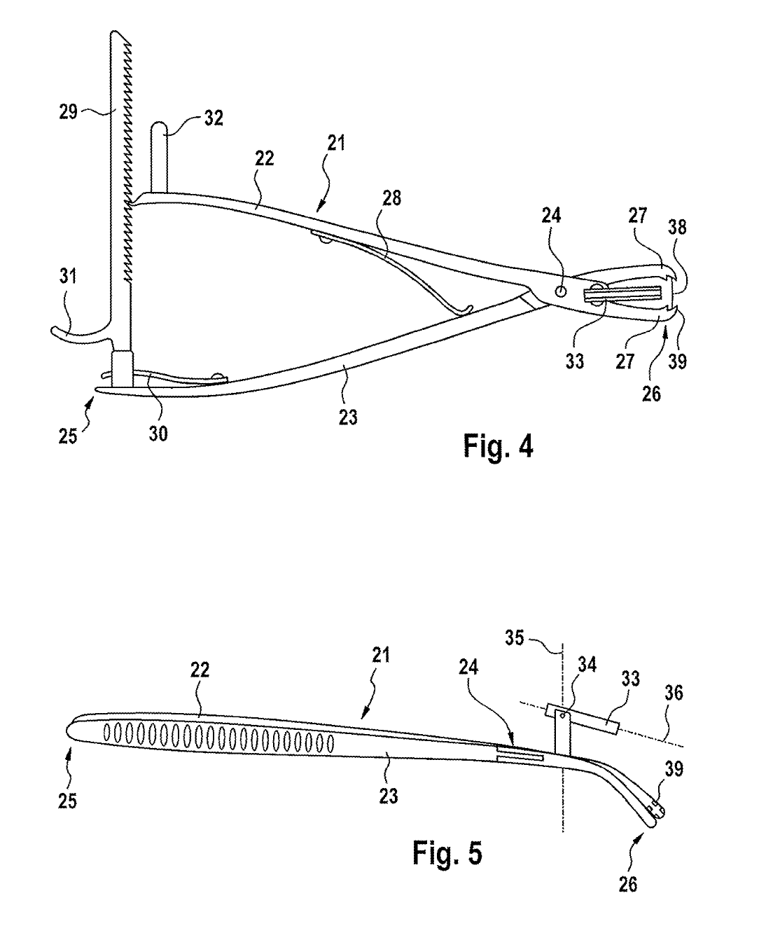

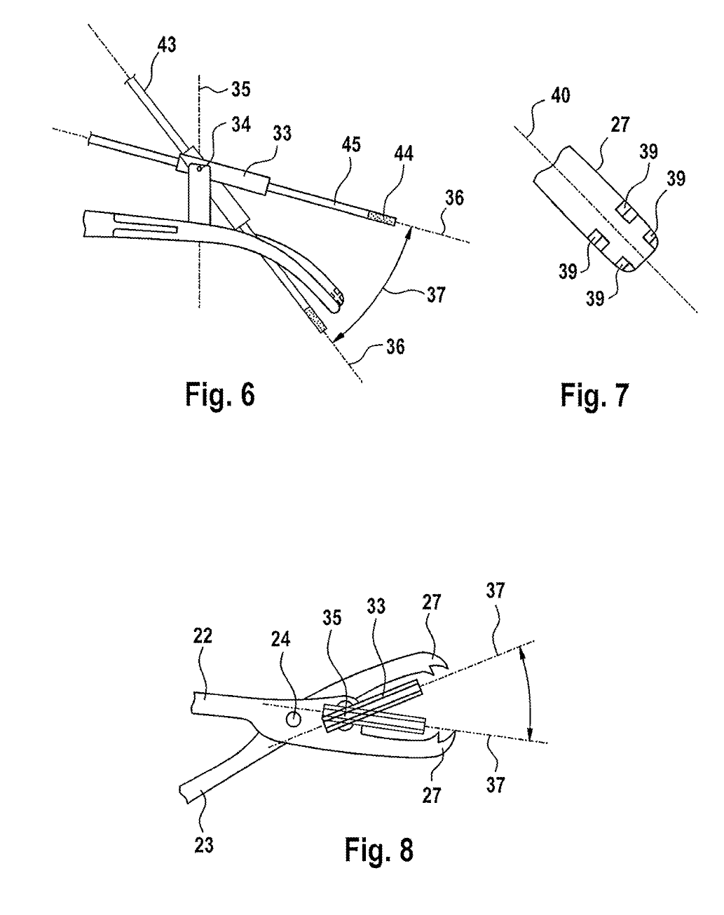

The surgical instrument set, as shown in FIGS. 4 and 5, composed of a guide instrument 21 and of a reamer 45, as shown in FIG. 6, is used to form the recesses 17 in the articular processes 14, 15, 18, 19. The guide instrument 21 shown in FIGS. 4 and 5 comprises two plier limbs 22, 23, which are connected to each other via a plier joint 24. The plier limbs 22, 23 extend from a proximal end 25 of the guide instrument 21 to a distal end 26. At the distal end 26, there are plier heads 27 with which the guide instrument 21 engages on the articular processes 14, 15, 18, 19. According to FIG. 7, each of the plier heads 27 comprises, on its inner face, four projections 39, which are oriented in the direction of the respectively opposite plier head 27 and therefore in the direction of the plier movement. The projections 39 are arranged on both sides of a longitudinal axis 40 extending through the plier head 27. When the articular processes 14, 15, 18, 19 are engaged by the plier heads 27, the projections 39 penetrate into the articular processes 14, 15, 18, 19. The penetration is made easier by the projections 39 having a shape tapering to a point.

The surgeon operates the guide instrument 21 from the proximal end 25. In particular, the surgeon presses the proximal ends of the plier limbs 22, 23 together against the force of a spring 28, in order to engage the articular processes 14, 15, 18, 19 with the plier heads 27. The guide instrument 21 is held in the tensioned state by a locking element 29. A spring 30 formed on the plier limb 23 holds the locking element 29 in engagement with the opposite plier limb 22. A grip element 31 on the locking element 29 is used to disengage the locking element 29. A projection 32 on the plier limb 22 avoids the surgeon coming into contact with the teeth of the locking element 29.

A guide rail 33 is arranged between the plier joint 24 and the plier heads 27, which guide rail 33 defines a guide axis 36, as shown in FIGS. 5 and 6. The guide rail 33 is pivotable about a first pivot axis 34 and about a second pivot axis 35 relative to the plier limbs 22, 23. The first pivot axis 34 is oriented perpendicular to the guide axis 36. The second pivot axis 35 is oriented substantially parallel to the axis of the plier head 24.

As shown in FIG. 6, the reamer 45 of the instrument set comprises a shaft 43 and a work head 44. According to FIG. 6, the guide rail 33 is designed such that it can receive the shaft 43 of the reamer 45. The axis of the reamer 45 is fixed by the guide rail 33 to the guide axis 36. When the reamer 45 rotates in the guide rail 33, the rotation axis corresponds to the guide axis 36. When the guide rail 33 is pivoted about the first pivot axis 34, the guide axis 36 sweeps across a work sector 37. The work sector 37 intersects an imaginary connecting line 38 between the plier heads 27. By pivoting the guide rail 33 about pivot axis 34, the work head 44 of the reamer 45 can thus be moved along a plane located between the plier heads 27. According to FIG. 8, by pivoting the guide rail 33 about the second pivot axis 35, the orientation of the work sector 37 between the plier heads 27 can be modified. The reamer 45 is movable in the longitudinal direction relative to the guide rail 33.

In order to form a recess 17 in the articular processes 14, 15, 18, 19, the surgeon positions the guide instrument 21 on an articular process and presses the proximal ends 25 of the guide instrument 21 together, such that the plier heads 27 clamp the articular process between them. The projections 39 penetrate into the bone tissue of the articular process. The approximate points of penetration are indicated in FIGS. 1 to 3 by reference sign 42. The longitudinal axis 40 of the plier head 27 extends in each case between two adjacent points 42. In each of FIGS. 1 and 2, when the articular processes 14, 15, 18, 19 are clamped between the plier heads 27, the articular processes 14, 15 and 18, 19, respectively, are at the same time brought into a defined position relative to each other.

When the guide rail 33 is in this way brought into a defined position and is limited to pivoting movements relative to the articular processes 14, 15, 18, 19, the surgeon inserts the reamer 45 into the guide rail 33. By pivoting about the second pivot axis 35, the work head 44 of the reamer 45 is positioned such that it is correctly sited between the plier heads 27. Thereafter, in the actual working step, the guide rail 33 is pivoted about the first pivot axis 34, and the work head 44 of the reamer 45, during this pivoting movement, removes material from the articular processes in order to form the recesses 17. By moving the reamer 45 longitudinally in the guide rail 33, it is possible to determine how deep the work head 44 of the reamer 45 engages in the articular process. In FIG. 1, the work sector 37 in which the work head 44 of the reamer 45 moves during the working is indicated by a broken line. In each of FIGS. 1 and 2, recesses 17 are simultaneously formed in two articular processes 14, 15 and 18, 19, respectively, in a single work step. A facet joint prosthesis 20, as shown in FIGS. 2 and 3, can be inserted into the recesses 17.

1. A guide instrument for use on the articular processes of vertebral bodies, comprising:
a first plier head and a second plier head, located at a distal end of the guide instrument, wherein the first plier head and the second plier head are movable relative to each other; and
a guide rail, which defines a guide axis for a reamer, wherein the guide rail is pivotable about a first pivot axis such that the guide axis, when moved about the first pivot axis, spans a work sector, and wherein the work sector intersects an imaginary connecting line between the plier heads.

2. The guide instrument according to claim 1, wherein the guide rail is pivotable about a second pivot axis.

3. The guide instrument according to claim 2, wherein the second pivot axis lies in the plane of the work sector.

4. The guide instrument according to claim 1, wherein the work sector spans an area defined by an angle relative to the first pivot axis that is greater than 15°.

5. The guide instrument according to claim 1, wherein the work sector spans an area defined by an angle relative to the first pivot axis that is greater than 30°.

6. The guide instrument according to claim 1, wherein the work sector spans an area defined by an angle relative to the first pivot axis that is greater than 45°.

7. The guide instrument according to claims 1, further comprising:
a first plier limb located at a proximal end of a portion of the guide instrument for which the first plier head comprises the distal end;
a second plier limb located at a proximal end of a portion of the guide instrument for which the second plier head comprises the distal end; and
a plier joint having an axis of rotation and connecting the first plier limb and the second plier limb, wherein the plier limbs are angled outward from a plane oriented parallel to the axis of rotation of the plier joint.

8. A guide instrument according to claim 1, wherein the plier heads are arranged on a first side and the first pivot axis is arranged on a second side in relation to a plane lying perpendicular to the axis of rotation of the plier joint and extending through the plier joint.

9. A guide instrument according to claim 1, further comprising:
a plurality of projections arranged on each of the plier heads on their respective inwardly opposing faces, the projections oriented in the direction of movement of the plier heads and positioned on both sides of a longitudinal axis extending through each of the plier heads.

10. An instrument set for working the articular processes of vertebral bodies, comprising:
a guide instrument according to claim 1; and
a reamer, the reamer having a shaft adapted to the guide rail.

11. The instrument set according to claim 8, wherein the shaft of the reamer is movable in the longitudinal direction in the guide rail.

12. A method for working the articular processes of vertebral bodies, comprising:
positioning a pivotable guide rail relative to the articular process such that a work sector spanned by the pivoting movement of the guide rail intersects the articular process;
fixing the guide rail relative to the articular process such that the guide rail is limited to pivoting movements;
inserting a reamer into the guide rail;
working the articular process with the reamer and at the same time pivoting the guide axis in order to form a recess in the articular process.

13. The method according to claim 10, wherein the guide rail is fixed relative to two articular processes.

14. The method according to claim 11, further comprising:
performing a pivoting movement with the reamer guided by the guide rail, wherein both articular processes are worked in a common work step.

15. The method according to claim 10, further comprising:
adjusting the depth of penetration of the reamer into the articular process by moving the reamer along the guide rail.
